(19) United States
(12) Patent Application Publication
(10) Pub. No.: US 2017/0151405 A1
(43) Pub. Date: Jun. 1, 2017

(54) TRACHEAL TUBE

(71) Applicant: TERUMO KABUSHIKI KAISHA, TOKYO (JP)

(72) Inventor: Anri FUJII, Kanagawa (JP)

(21) Appl. No.: 15/331,580

(22) Filed: Oct. 21, 2016

(30) Foreign Application Priority Data
Nov. 19, 2015 (JP) 2015-226639

(51) Int. Cl. A61M 16/04

(52) U.S. Cl. CPC A61M 16/0445; A61M 16/0486; A61M 16/0463

(57) ABSTRACT

Disclosed herein is a tracheal tube including a tube main body and a cuff that is mounted to the tube main body and has a barrel portion capable of being deflated and inflated. A covering region that covers a tracheal inner wall of a trachea in a state in which the barrel portion is inflated in the trachea has a contact part that gets contact with the tracheal inner wall and a wrinkle part that does not get contact with the tracheal inner wall, and the wrinkle part includes a first wrinkle and a second wrinkle that extends in a direction intersecting a predetermined part of the first wrinkle and has a termination part that does not intersect the predetermined part and terminates at such a position as to sandwich the contact part with the predetermined part.

### CROSS REFERENCE TO RELATED APPLICATIONS

This application is based on and claims priority to Japanese Application No. JP2015-226639, filed on Nov. 19, 2015, the entire contents of which are incorporated herein by reference.

### BACKGROUND

The present disclosure relates to a tracheal tube.

As a related art, a tracheostomy tube is known as a tracheal tube that directly connects the outside of a body and the inside of a trachea to facilitate breathing for a patient for which spontaneous breathing is difficult. To the tracheostomy tube, a cuff that can be deflated and inflated is attached on the outer circumferential surface of the tube main body. The tracheostomy tube is inserted into the trachea of the patient, with the cuff deflated, and then the cuff is inflated. This causes the cuff to get contact with the tracheal inner wall, so that the tracheostomy tube is indwelled in the trachea (for example, refer to Japanese Patent No. 5271898 and Japanese Patent Laid-Open No. 2000-167060).

### SUMMARY

When the cuff is inflated in the trachea of a patient and the tracheal tube is used, foreign objects such as sputum, saliva, blood, and accidentally-swallowed object accumulate on the trachea upstream side (on the jaw side). The foreign objects that accumulate can be removed by being sucked from a lumen made in the tracheal tube for example. However, in some cases, wrinkles are formed in the outer surface of the cuff when the cuff is inflated in the trachea and a gap due to the wrinkle is generated between the outer surface of the cuff and the tracheal inner wall of the patient. The generation of the gap due to the wrinkle causes a possibility that a foreign object flows from this gap toward the trachea downstream side to flow into the lung. Due to the foreign object flowing into the lung in this manner, the risk that pneumonia develops in the patient increases.

An object of the present disclosure is to provide a tracheal tube that allows improvement in safety in view of the aforesaid problem.

A tracheal tube as a first aspect of the present disclosure includes a tube main body and a cuff that is mounted to the tube main body and has a barrel portion capable of being deflated and inflated. A covering region that covers a tracheal inner wall of a trachea in a state in which the barrel portion is inflated in the trachea has a contact part that gets contact with the tracheal inner wall and a wrinkle part that does not get contact with the tracheal inner wall. The wrinkle part includes a first wrinkle and a second wrinkle that extends in a direction intersecting a predetermined part of the first wrinkle and has a termination part that does not intersect the predetermined part and terminates at such a position as to sandwich the contact part with the predetermined part.

As one embodiment of the present disclosure, the termination part can be configured to extend toward a circumferential direction of the tube main body relative to an extension direction at the predetermined part of the first wrinkle.

As one embodiment of the present disclosure, at least one end of the first wrinkle reaches a distal rim or a proximal rim of the covering region.

As one embodiment of the present disclosure, the termination part can be configured to extend toward the center axis line direction of the tube main body relative to the extension direction at the predetermined part of the first wrinkle.

As one embodiment of the present disclosure, one end of the second wrinkle reaches a distal rim or a proximal rim of the covering region.

As one embodiment of the present disclosure, the minimum width of the contact part between the first wrinkle and the termination part of the second wrinkle is smaller than 1 mm.

As one embodiment of the present disclosure, the contact part has a sealing part that gets contact with the tracheal inner wall seamlessly in a circumferential direction of the barrel portion, and the width of at least part of the sealing part is smaller than 1 mm.

As one embodiment of the present disclosure, the barrel portion is rotationally symmetric about a predetermined axis in the inflated state and has a taper part whose diameter around the predetermined axis increases from one end side of the predetermined axis toward the other end side.

According to the present disclosure, a tracheal tube that allows improvement in safety can be provided.

### BRIEF DESCRIPTION OF THE DRAWINGS

FIG. 1 is a diagram illustrating the state in which a tracheal tube according to one embodiment of the present disclosure is indwelled in a trachea;

FIG. 2 is a perspective view illustrating solely a tube main body in the tracheal tube illustrated in FIG. 1;

FIG. 3 is a side view illustrating the outline of a cuff of the tracheal tube illustrated in FIG. 1;

FIG. 4 is a sectional view illustrating the outline of the cuff of the tracheal tube illustrated in FIG. 1;

FIG. 5 is a diagram illustrating one example of a covering region recorded on a laminate film, obtained by an experiment using the tracheal tube having the cuff illustrated in FIG. 3 and FIG. 4;

FIG. 6 is a sectional view illustrating the state in which a cuff as a modification example of the cuff illustrated in FIG. 3 and FIG. 4 is inflated;

FIG. 7 is a sectional view illustrating the state in which the cuff illustrated in FIG. 6 is in the middle of inflation;

FIG. 8A is a sectional view illustrating the state in which a cuff as another modification example of the cuff illustrated in FIG. 3 and FIG. 4 is in the middle of inflation, and FIG. 8B is a sectional view illustrating the state in which a cuff 3 as yet another modification example of the cuff illustrated in FIG. 3 and FIG. 4 is in the middle of inflation;

FIG. 9A is a diagram illustrating an exemplary embodiment of the shape of the cuff, and FIG. 9B is a diagram illustrating an exemplary embodiment of the shape of the cuff; and

FIG. 10 is a flowchart illustrating a mounting method of the cuff illustrated in FIG. 6 and FIG. 7.

### DETAILED DESCRIPTION OF THE PREFERRED EMBODIMENT

A tracheal tube according to one embodiment of the present disclosure will be described below with reference to the drawings. A component common in the respective drawings is given the same symbol.

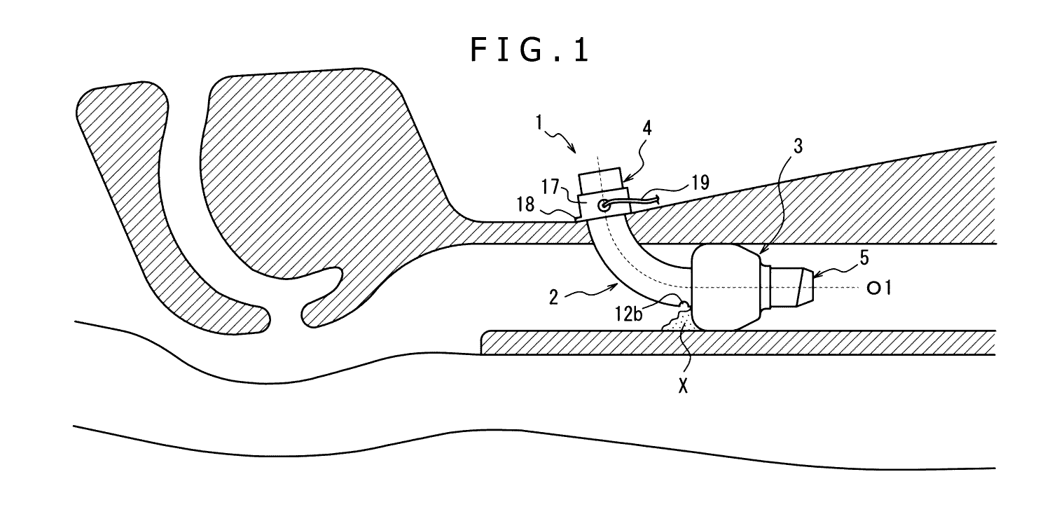

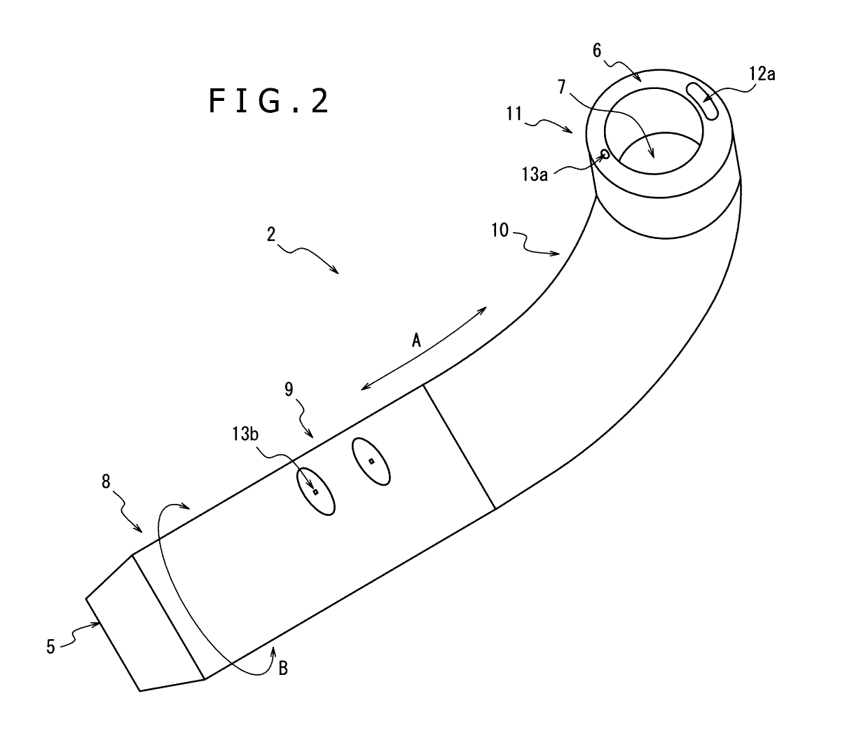

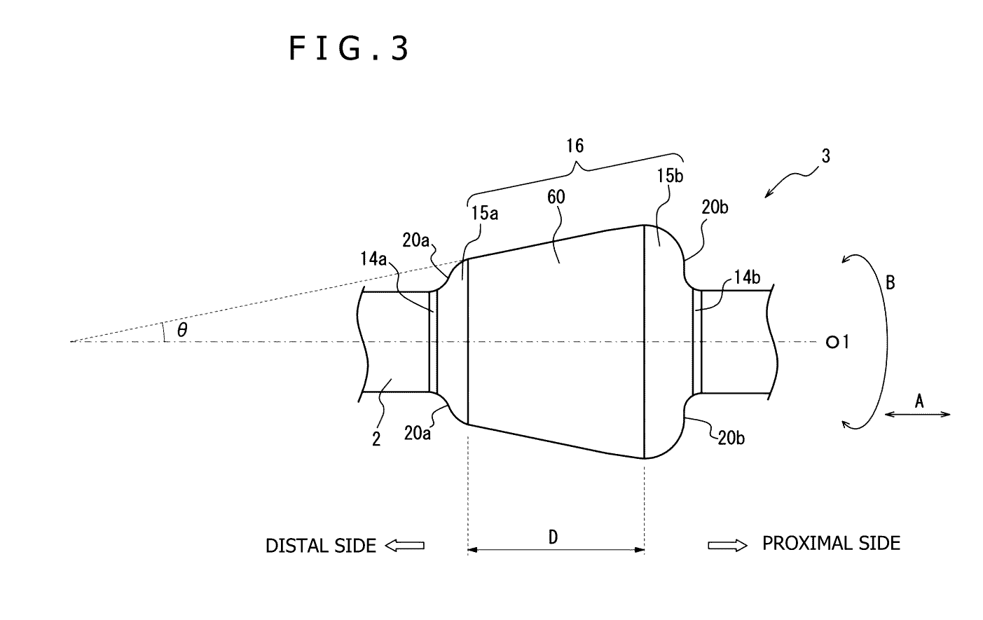

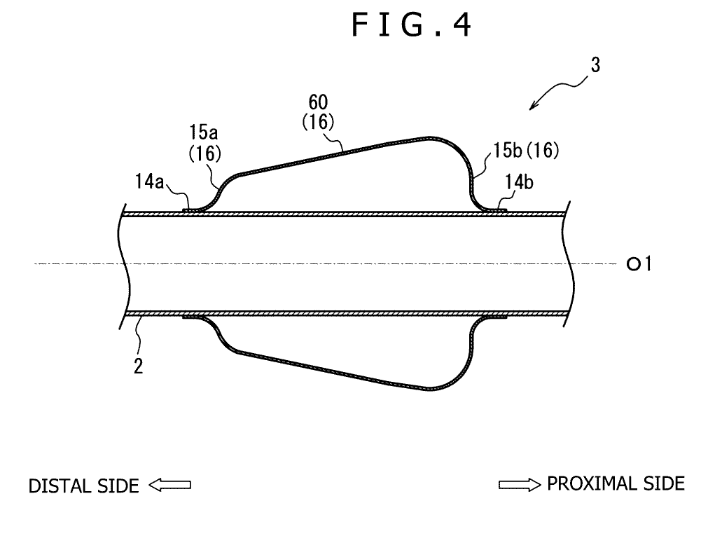

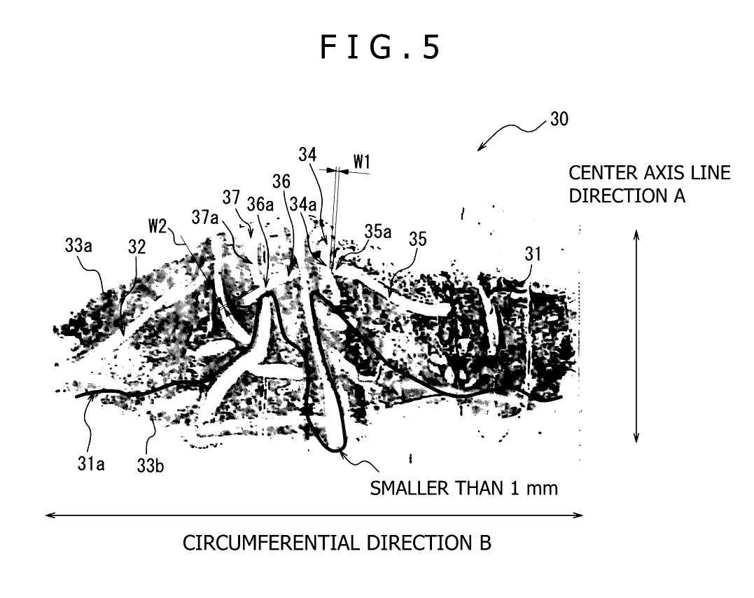

FIG. 1 is a diagram illustrating the state in which the tracheal tube according to the one embodiment of the present disclosure is indwelled in a trachea. FIG. 2 is a perspective view illustrating solely a tube main body 2 in a tracheal tube 1 illustrated in FIG. 1. FIG. 3 and FIG. 4 are a side view and a sectional view illustrating the outline of a cuff 3 of the tracheal tube 1 illustrated in FIG. 1. FIG. 5 is a diagram illustrating one example of a covering region recorded on a laminate film, obtained by an experiment using the tracheal tube 1 having the cuff 3 illustrated in FIG. 3 and FIG. 4.

As illustrated in FIG. 1, the tracheal tube 1 includes the tube main body 2, the cuff 3 that is attached onto the outer circumferential surface of this tube main body 2 and can be deflated and inflated, and a flange member 4 mounted to one end part of the tube main body 2.

In the tube main body 2, a hollow part 7 that penetrates from a distal end 5 to a proximal end 6 in the extension direction of a center axis line O1 (hereinafter, referred to simply as “center axis line direction A”) of the outer circumferential surface of the tube main body 2 is marked out. In the state in which the tracheal tube 1 is inserted from the outside into the trachea and is indwelled, an airway is ensured by this hollow part 7. Note that the distal end 5 of the tube main body 2 is the distal end of the tube main body 2 and is one end located on the tracheal bifurcation side in the state in which the tracheal tube 1 is indwelled in the trachea. Furthermore, the proximal end 6 is the proximal end of the tube main body 2 and is the other end located on the jaw side in the state in which the tracheal tube 1 is indwelled in the trachea.

The tube main body 2 includes a distal portion 8 including the distal end 5, a cuff-mounted portion 9 that is continuous on the side of the proximal end 6 in the distal portion 8 in the center axis line direction A and has an outer circumferential surface onto which the cuff 3 is attached, a curving portion 10 that is continuous on the side of the proximal end 6 in this cuff-mounted portion 9, and a proximal portion 11 that is continuous on the side of the proximal end 6 in this curving portion 10 and includes the proximal end 6. In other words, the distal portion 8 of the tube main body 2 is linked to the proximal portion 11 with the intermediary of the cuff-mounted portion 9 and the curving portion 10. Note that the flange member 4 is mounted to the proximal portion 11.

In the wall of the tube main body 2 between the outer circumferential surface of the tube main body 2 and the inner circumferential surface that marks out the hollow part 7 of the tube main body 2, two hollow parts that extend along the center axis line O1 are marked out. Specifically, the tube main body 2 has a first lumen and a second lumen that are formed in the wall and extend along the center axis line O1 from a first proximal opening 12a and a second proximal opening 13a marked out in the proximal surface. Note that, although the first lumen and the second lumen that are marked out in the wall and have small diameters are also hollow parts, they are referred to as “lumen” here for differentiation from the large-diameter hollow part 7 for ensuring an airway for convenience of description.

The first lumen extends from the first proximal opening 12a in the proximal surface to a predetermined position on the side of the proximal portion 11 relative to the cuff 3 and the cuff-mounted portion 9, and communicates with the outside of the tube main body 2 through a suction port 12b that is formed at the predetermined position and penetrates to the outer circumferential surface of the tube main body 2. Note that the suction port 12b in the present embodiment is a suction port and is formed in the curving portion 10 as the position on the side of the proximal portion 11 relative to the cuff 3 and the cuff-mounted portion 9. The first lumen sucks and removes foreign objects X such as sputum, saliva, blood, and accidentally-swallowed object that accumulate on the trachea upstream side (on the jaw side) relative to the cuff 3 in the state of being indwelled in the trachea.

The second lumen extends from the second proximal opening 13a in the proximal surface to the position of the cuff 3 and the cuff-mounted portion 9, and communicates with the outside through a flow path 13b that is formed at the position and penetrates to the outer circumferential surface of the tube main body 2. Therefore, for example by using a syringe or the like, fluid such as air is supplied into the space (annular space) marked out by the outer circumferential surface of the cuff-mounted portion 9 and the inner surface of the cuff 3 from the second proximal opening 13a of the second lumen through the flow path 13b. Thereby, the cuff 3 can be inflated by this supplied fluid. Furthermore, for the cuff 3 in the inflated state, the fluid is sucked from the annular space through the flow path 13b and the second proximal opening 13a of the second lumen. This can deflate the cuff 3. As above, the second lumen is a lumen used for deflating and inflating the cuff 3 and will be referred to as the “cuff lumen” hereinafter.

As the constituent material of the tube main body 2, the following various kinds of resin can be used for example: silicone, polyvinyl chloride, polyethylene, polypropylene, cyclic polyolefin, polystyrene, poly-(4-methylpentene-1), polycarbonate, acrylic resin, acrylonitrile-butadiene-styrene copolymer, polyester such as polyethylene terephthalate, butadiene-styrene copolymer, and polyamide (for example, nylon 6, nylon 6/6, nylon 6/10, or nylon 12). It is preferable to use, among them, a resin such as polyvinyl chloride, polypropylene, cyclic polyolefin, polyester, or poly-(4-methylpentene-1) in terms of easiness of shaping.

The flange member 4 is mounted to the proximal portion 11 (see FIG. 2 and so forth) of the tube main body 2 as illustrated in FIG. 1. When the tube main body 2 is inserted from the outside of the body into the trachea and the tracheal tube 1 is indwelled, the flange member 4 abuts against skin to fix the distal portion 8 at a proper position in the trachea. The proximal portion 11 of the tube main body 2 is internally inserted into the flange member 4. The flange member 4 includes a tubular part 17 that is mounted to the tube main body 2 by being fit to the tube main body 2 and has a circular cylindrical shape and a plate-shaped flange part 18 that protrudes from the outer wall of this tubular part 17 toward the outside in the radial direction and abuts against skin in the state in which the tracheal tube 1 is indwelled.

The first lumen communicates with the outside of the tracheal tube 1 on the proximal side of the tracheal tube 1 through a corresponding communication hole formed in the tubular part 17. Suction of the foreign object X such as an accidentally-swallowed object is carried out by connecting a syringe, a suction pump, or the like to one end of a suction tube 19 having the other end fit to the communication hole of the tubular part 17 exposed outside the body and performing suction.

Furthermore, the cuff lumen communicates with the outside of the tracheal tube 1 on the proximal side of the tracheal tube 1 through a corresponding communication hole formed in the tubular part 17. Therefore, if a syringe or the like is connected to one end of a tube for the cuff having the other end fit to the communication hole of the tubular part 17 exposed outside the body, supply and suction of fluid to and from the annular space of the cuff 3 can be carried out by operation of the syringe or the like existing outside the body and thereby inflation and deflation of the cuff 3 can be operated.

The flange member 4 can be formed by a material similar to that of the tube main body 2 for example.

The cuff 3 is attached onto the outer circumferential surface of the cuff-mounted portion 9 of the tube main body 2 and can be inflated through the above-described cuff lumen of the tube main body 2 by the pressure of the fluid supplied into the annular space marked out by the outer circumferential surface of the cuff-mounted portion 9 and the inner surface of the cuff 3. Therefore, in attempting to insert the tracheal tube 1 from the outside into the trachea and indwell the tracheal tube 1 at a predetermined position, the fluid is supplied into the annular space through the cuff lumen of the tube main body 2 and the cuff 3 is inflated. Thereby, the outer surface of the inflated cuff 3 is brought into tight contact with the tracheal inner wall and the cuff 3 is clamped by the inner circumferential wall surface of the trachea by the friction force between the outer surface of the cuff 3 and the tracheal inner wall, and so forth. In addition, the cuff 3 occludes the trachea around the tube main body 2. For this reason, the position of the cuff 3 in the trachea is fixed and the tracheal tube 1 can be indwelled at the above-described predetermined position.

Furthermore, in withdrawing the tracheal tube 1 from the inside of the trachea to the outside and in adjusting the position at which the tracheal tube 1 is indwelled, and so forth, the fluid in the annular space is sucked by using the cuff lumen and the cuff 3 is deflated. This allows the tube main body 2 of the tracheal tube 1 to move in the trachea along the trachea.

In the cuff 3, the end edge part on the side of the proximal end 6 in the center axis line direction A (corresponding to a proximal connected part 14b to be described later) and the end edge part on the side of the distal end 5 (corresponding to a distal connected part 14a to be described later) are each joined onto the outer circumferential surface of the cuff-mounted portion 9 by welding, high-frequency fusion bonding, or the like across the whole region of a circumferential direction B (see FIG. 2) of the tube main body 2. This forms the above-described annular space by the inner surface of the cuff 3 and the outer circumferential surface of the cuff-mounted portion 9.

A more detailed description will be made below about the cuff 3 with reference to FIG. 3 and FIG. 4. In FIG. 3 and FIG. 4, only the cuff 3 and a part near the cuff 3 in the tube main body 2 to which the cuff 3 is attached are illustrated and the state in which the cuff 3 is inflated at a certain pressure (e.g. 16 to 25 mmHg) is illustrated. That is, the cuff 3 becomes the state illustrated in FIG. 3 and FIG. 4 when being inflated without being inserted into a trachea. Furthermore, representation of the lumens is omitted in the section including the center axis line O1 illustrated in FIG. 4.

The cuff 3 includes a barrel portion 16 that can be deflated and inflated in the radial direction of the tube main body 2, and the distal connected part 14a on the side of the distal end 5 of the tube main body 2 and the proximal connected part 14b on the side of the proximal end 6 of the tube main body 2 that are continuous on both sides of this barrel portion 16 in the center axis line direction A and are fixed to the outer wall of the tube main body 2.

The outer shape of the barrel portion 16 is a substantially conical trapezoid shape that has the distal connected part 14a and the proximal connected part 14b on the upper base side and the lower base side, respectively, and has the center axis line O1 as the center axis. Specifically, the barrel portion 16 includes the following parts: a taper part 60 that is substantially rotationally symmetric about the center axis line O1 and in which the diameter of the outer surface (outer diameter) increases from the side of the distal end 5 toward the side of the proximal end 6, with the center axis line O1 being the center of the diameter; an annular distal coupling part 15a that is continuous with the side of the distal end 5 in this taper part 60 and couples the taper part 60 and the distal connected part 14a; and an annular proximal coupling part 15b that is continuous with the side of the proximal end 6 in the taper part 60 and couples the taper part 60 and the proximal connected part 14b.

As illustrated in FIG. 3 and FIG. 4, the taper part 60 is inclined with respect to the center axis line O1 in such a manner as to come closer to the center axis line O1 from the side of the proximal end 6 toward the side of the distal end 5. More specifically, as illustrated in FIG. 3 and FIG. 4, the taper part 60 is inclined in a straight line manner with respect to the center axis line O1 in side view and sectional view, and the angle formed by the taper part 60 and the center axis line O1 illustrated in FIG. 3 and FIG. 4 (taper angle) 8 is 12°.

The distal connected part 14a and the proximal connected part 14b are parts fixed to the outer wall of the tube main body 2 by fusion bonding or the like in the cuff 3. Here, the distal connected part 14a and the proximal connected part 14b illustrated in FIG. 3 and FIG. 4 are each fixed to the outer wall of the tube main body 2 in the state of extending to the outside relative to the barrel portion 16 in the center axis line direction A. Specifically, the distal connected part 14a is fixed to the outer wall of the tube main body 2 in the state of extending to the side of the distal end 5 relative to the barrel portion 16 in the center axis line direction A, and the proximal connected part 14b is fixed to the outer wall of the tube main body 2 in the state of extending to the side of the proximal end 6 relative to the barrel portion 16 in the center axis line direction A.

As described above, the taper part 60 of the barrel portion 16 and the distal connected part 14a are coupled by the distal coupling part 15a of the barrel portion 16. Furthermore, the taper part 60 of the barrel portion 16 and the proximal connected part 14b are coupled by the proximal coupling part 15b of the barrel portion 16. As illustrated in FIG. 3, the outer wall of the distal coupling part 15a forms a gentle curved surface having an inflection point 20a so as to smoothly couple the outer wall of the taper part 60 and the outer wall of the distal connected part 14a when the barrel portion 16 is inflated. Furthermore, the outer wall of the proximal coupling part 15b forms a gentle curved surface having an inflection point 20b so as to smoothly couple the outer wall of the taper part 60 and the outer wall of the proximal connected part 14b when the barrel portion 16 is inflated.

In the barrel portion 16, length D of the taper part 60 along the center axis line O1 can be decided as appropriate and is set to 22 to 26 mm in the present embodiment. Furthermore, the maximum diameter (cuff diameter) of the circle formed by the outer surface of the barrel portion 16 in a surface orthogonal to the center axis line O1 can be decided as appropriate according to the size of the trachea of a patient for example and is set to 26 mm in the present embodiment. Note that it is preferable that this maximum diameter be such a diameter that, when the cuff 3 is inflated, the tracheal tube 1 is indwelled in the trachea of a patient due to application of a predetermined proper pressure to the tracheal inner wall from the outer surface of the cuff 3.

The material of the membrane forming the outer surface of the cuff 3 in the present embodiment is soft polyvinyl chloride and has a Young's modulus of 0.34 MPa. Note that, as the material of the membrane forming the outer surface of the cuff 3, polyurethane, polyethylene, polypropylene, polyester, ethylene-vinyl acetate copolymer (EVA), silicone, or a material that is obtained by mixing any materials among them and has flexibility may be used for example. Furthermore, the membrane forming the outer surface of the cuff 3 in the present embodiment has a membrane thickness of 0.06 mm.

Next, an experiment made by the inventor with use of the tracheal tube 1 according to the present embodiment will be described. The inventor used the tracheal tube 1 according to the present embodiment for the trachea of a subject and made the experiment relating to the state of the contact between the cuff 3 and the tracheal inner wall of the subject in the use state.

Specifically, first the inventor stuck a laminate film to the inner surface of the trachea by inserting the laminate film turned to a tubular shape into the trachea of the subject (test body) and to the position with which the inflated cuff 3 gotten contact when the tracheal tube 1 was used. As the laminate film, a film (thickness 0.1 mm) exclusively for the laminator, made by ASKA CORPORATION, was used. Next, the tracheal tube 1 was inserted into the trachea of the subject in the state in which the cuff 3 on which paint was applied on the outer surface was deflated, and the cuff 3 was inflated at a certain pressure (e.g. 16 to 25 mmHg). At this time, the paint applied on the outer surface of the cuff 3 adheres to the part in contact with the outer surface of the barrel portion 16 of the cuff 3 in the laminate film stuck to the inner surface of the trachea. Besides, the part at which a wrinkle is generated in the outer surface of the barrel portion 16 of the cuff 3 does not get contact with the laminate film and therefore the paint on this part does not adhere to the laminate film. Then, the cuff 3 was deflated and the tracheal tube 1 was withdrawn from the trachea. Thereafter, the laminate film was peeled off from the trachea of the subject. This experiment was made on plural subjects.

FIG. 5 is a diagram illustrating one example of the covering region recorded on the laminate film, obtained by the above-described experiment. FIG. 5 illustrates the laminate film that was withdrawn from the trachea of the subject and then was cut along the center axis line direction A to be extended. Here, a covering region 30 is a region that covers the tracheal inner wall in the state in which the barrel portion 16 is inflated in the trachea of the subject, in the outer wall of the barrel portion 16. The covering region 30 has a contact part 31 as the part that gets contact with the tracheal inner wall in the barrel portion 16 and a wrinkle part 32 that covers the tracheal inner wall but does not get contact with the tracheal inner wall.

The contact part 31 is the part that gets contact with the tracheal inner wall in the barrel portion 16 and therefore appears as a region to which the paint adheres in the laminate film illustrated in FIG. 5. The contact part 31 is a region where the barrel portion 16 is brought into tight contact with the tracheal inner wall and thus a foreign object such as an accidentally-swallowed object does not flow into the contact part 31.

The wrinkle part 32 is a region that does not get contact with the tracheal inner wall and therefore appears as a region to which the paint does not adhere in the laminate film illustrated in FIG. 5. The wrinkle part 32 is formed due to that the outer surface of the barrel portion 16 is not fully inflated and wrinkles are formed in the outer surface of the barrel portion 16. Therefore, the wrinkle part 32 has a space between the barrel portion 16 and the tracheal inner wall. For this reason, there is a possibility that a foreign object such as an accidentally-swallowed object flows into the wrinkle part 32.

As illustrated in FIG. 5, the contact part 31 extends across the whole of the laminate film in the circumferential direction B. In particular, the contact part 31 of the covering region 30 illustrated in FIG. 5 has a sealing part 31a that gets contact with the tracheal inner wall seamlessly in the circumferential direction B of the barrel portion 16. In FIG. 5, one example of the seamless sealing part 31a is illustrated by a heavy line. The seamless sealing part 31a formed in the contact part 31 of the covering region 30 is not limited to the sealing part 31a identified by the path illustrated by the heavy line in FIG. 5 and may be any as long as it is the sealing part 31a identified by a seamless path continuous in the circumferential direction B in the contact part 31. That is, the seamless sealing part 31a means a place where unicursal drawing is possible in the circumferential direction B in the contact part 31.

Due to that the contact part 31 of the covering region 30 has the seamless sealing part 31a as above, the contact part 31, into which a foreign object does not flow, is continuously formed seamlessly in the circumferential direction B. This eliminates the flowing of a foreign object from the proximal side of the cuff 3 (on the side of the proximal end 6 of the tube main body 2) into the distal side (on the side of the distal end 5 of the tube main body 2). For this reason, the risk of the flowing of the foreign object into the lung is reduced and the risk that pneumonia develops in the patient is reduced. In this manner, the safety of the tracheal tube 1 is enhanced.

Furthermore, the inventor made strenuous studies about a configuration in which the above-described sealing part 31a is easily formed when the barrel portion 16 is inflated in a trachea. As a result, the inventor has reached the discovery that the formation of the sealing part 31a is facilitated by inducing the shape and the extension direction of the wrinkle of the wrinkle part 32 to become a predetermined shape and a predetermined extension direction and forming the wrinkle part 32 under this predetermined condition.

More specifically, as a result of carrying out the above-described experiment on plural subjects, the inventor has reached the discovery that the sealing part 31a is easily formed if the wrinkle part 32 of the covering region 30 is configured to include a first wrinkle and a second wrinkle that extends in a direction intersecting a predetermined part of this first wrinkle and has a termination part that does not intersect the predetermined part but terminates at such a position as to sandwich the contact part with the predetermined part. In other words, the termination part of the second wrinkle exists near an extending part that linearly extends as the predetermined part of the first wrinkle. Such a relationship of the shape and the extension direction between the first wrinkle and the second wrinkle readily occurs due to interference between the first wrinkle and the second wrinkle. Specifically, the relationship readily occurs when the second wrinkle interferes with the first wrinkle in such a manner as to narrow the first wrinkle or when the first wrinkle interferes with the second wrinkle in such a manner as to split the second wrinkle. If such a relationship between the wrinkles is realized, formation of a penetrating wrinkle that penetrates the covering region 30 in the center axis line direction A is impeded. That is, formation of the above-described sealing part 31a in the contact part 31 is facilitated.

A description will be made below about each of the relationship between the first and second wrinkles in which the second wrinkle readily interferes with the first wrinkle in such a manner as to narrow the first wrinkle (hereinafter, referred to as the “relationship between wrinkles of the narrowing type”) and the relationship between the first and second wrinkles in which the first wrinkle readily interferes with the second wrinkle in such a manner as to split the second wrinkle (hereinafter, referred to as the “splitting type”).

<Relationship Between Wrinkles of Narrowing Type>

First, a description will be made about the case in which the termination part in the above-described second wrinkle extends toward the circumferential direction B relative to the extension direction at a predetermined part of the first wrinkle. In other words, this case is the case in which the predetermined part of the first wrinkle extends toward the center axis line direction A compared with the termination part of the second wrinkle relatively. As one example of the case, the case is cited in which the first wrinkle is a wrinkle that extends in the center axis line direction A (vertical wrinkle), including the predetermined part, and the second wrinkle is a wrinkle that extends in the circumferential direction B (horizontal wrinkle), including the termination part.

In FIG. 5, a wrinkle indicated by numeral “34” is one example of the above-described first wrinkle and a wrinkle indicated by numeral “35” is one example of the above-described second wrinkle. In the following, the wrinkles indicated by numerals “34” and “35” in FIG. 5 will be deemed as the first wrinkle 34 and the second wrinkle 35 and details of the first wrinkle 34 and the second wrinkle 35 will be described.

As illustrated in FIG. 5, the first wrinkle 34 extends in a direction substantially parallel to the center axis line direction A. In other words, the first wrinkle 34 has a part that extends in the direction substantially parallel to the center axis line direction A as a predetermined part 34a.

The second wrinkle 35 has a termination part 35a that extends in a direction intersecting the predetermined part 34a of the first wrinkle 34, i.e. the circumferential direction B. The termination part 35a does not intersect the predetermined part 34a of the first wrinkle 34 but terminates at such a position as to sandwich part of the contact part 31 with the predetermined part 34a.

If the first wrinkle 34 and the second wrinkle 35 are in such a relationship, the second wrinkle 35 interferes with the first wrinkle 34 in such a manner as to narrow the first wrinkle 34 in many cases. In the example illustrated in FIG. 5, part of the first wrinkle 34 as the predetermined part 34a has a slightly-bent shape in such a manner as to be pushed and bent in the circumferential direction B by the termination part 35a of the second wrinkle 35. In this manner, due to the effect of the pushing of the predetermined part 34a of the first wrinkle 34 in the circumferential direction B by the termination part 35a of the second wrinkle 35, the predetermined part 34a of the first wrinkle 34 is bent or narrowed as illustrated in FIG. 5. As a result, the first wrinkle 34 readily terminates on one side in the extension direction of the first wrinkle 34 with respect to this predetermined part 34a.

In particular, if one end of the first wrinkle 34 reaches a distal rim 33a that is the rim on the distal side in the center axis line direction A in the covering region 30 or a proximal rim 33b that is the rim on the proximal side in the center axis line direction A (if the one end of the first wrinkle 34 communicates with the outside of the covering region 30 from the distal rim 33a or the proximal rim 33b), the other end side of the first wrinkle 34 is readily made to terminate in the covering region 30 and formation of the penetrating wrinkle that penetrates the covering region 30 in the center axis line direction A is impeded if the above-described state in which the predetermined part 34a is subject to interference by the termination part 35a of the second wrinkle 35 can be realized. In the example illustrated in FIG. 5, one end of the first wrinkle 34 on the side of the distal end 5 reaches the distal rim 33a of the covering region 30 whereas the other end of the first wrinkle 34 on the side of the proximal end 6 terminates in the covering region 30 at a position on the side of the proximal end 6 relative to the predetermined part 34a.

<Relationship Between Wrinkles of Splitting Type>

Next, a description will be made about the case in which the termination part in the above-described second wrinkle extends toward the center axis line direction A relative to the extension direction at a predetermined part of the first wrinkle. In other words, this case is the case in which the predetermined part of the first wrinkle extends toward the circumferential direction B compared with the termination part of the second wrinkle relatively. As one example of the case, the case is cited in which the first wrinkle is a wrinkle that extends in the circumferential direction B (horizontal wrinkle), including the predetermined part, and the second wrinkle is a wrinkle that extends in the center axis line direction A (vertical wrinkle), including the termination part.

In FIG. 5, a wrinkle indicated by numeral “36” is one example of the above-described first wrinkle and a wrinkle indicated by numeral “37” is one example of the above-described second wrinkle. In the following, the wrinkles indicated by numerals “36” and “37” in FIG. 5 will be deemed as the first wrinkle 36 and the second wrinkle 37 and details of the first wrinkle 36 and the second wrinkle 37 will be described.

As illustrated in FIG. 5, the second wrinkle 37 extends in a direction substantially parallel to the center axis line direction A. More specifically, the second wrinkle 37 has a termination part 37a that extends in a direction intersecting a predetermined part 36a of the first wrinkle 36, i.e. the center axis line direction A. The termination part 37a does not intersect the predetermined part 36a of the first wrinkle 36 but terminates at such a position as to sandwich part of the contact part 31 with the predetermined part 36a.

As illustrated in FIG. 5, the first wrinkle 36 extends toward the circumferential direction B relative to the extension direction of the termination part 37a of the second wrinkle 37. In other words, the first wrinkle 36 has the predetermined part 36a that extends toward the circumferential direction B relative to the extension direction of the termination part 37a of the second wrinkle 37.

If the first wrinkle 36 and the second wrinkle 37 are in such a relationship, the first wrinkle 36 interferes with the second wrinkle 37 in such a manner as to split the second wrinkle 37 in many cases. In the example illustrated in FIG. 5, the second wrinkle 37 is split by the predetermined part 36a of the first wrinkle 36 and terminates by the termination part 37a.

In particular, if one end of the second wrinkle 37 reaches the distal rim 33a, which is the rim on the distal side in the center axis line direction A in the covering region 30, or the proximal rim 33b, which is the rim on the proximal side in the center axis line direction A (if the one end of the second wrinkle 37 communicates with the outside of the covering region 30 from the distal rim 33a or the proximal rim 33b), by causing the predetermined part 36a of the first wrinkle 36 to be located opposed to the extension direction of the second wrinkle 37 on the other end side of the second wrinkle 37, the extension of the other end side of the second wrinkle 37 in the center axis line direction A is inhibited and the other end side of the second wrinkle 37 is easily made to become the termination part 37a that terminates in the covering region 30 as illustrated in FIG. 5. That is, it is difficult for the second wrinkle 37 to become the penetrating wrinkle that penetrates the covering region 30 in the center axis line direction A due to the first wrinkle 36. That is, if such a relationship between the wrinkles is realized, formation of the sealing part 31a in the contact part 31 is facilitated.

As described above, if the wrinkle part 32 of the covering region 30 has a configuration including the first wrinkle and the second wrinkle that belong to one of the above-described narrowing type and splitting type, formation of the penetrating wrinkle that penetrates the covering region 30 in the center axis line direction A is impeded and formation of the sealing part 31a in the contact part 31 is facilitated.

Minimum width W1 of the contact part 31 located between the predetermined part 34a of the above-described first wrinkle 34 and the termination part 35a of the second wrinkle 35 is smaller than 1 mm. Furthermore, minimum width W2 of the contact part 31 located between the predetermined part 36a of the above-described first wrinkle 36 and the termination part 37a of the second wrinkle 37 is also smaller than 1 mm. If the minimum widths W1 and W2 of the contact part 31 sandwiched between the first wrinkle and the second wrinkle are smaller than 1 mm as above, the degree of interference between the first wrinkle and the second wrinkle is enhanced and the possibility that one wrinkle is bent, narrowed, split, or the like in the covering region can be enhanced.

Furthermore, the seamless sealing part 31a illustrated in FIG. 5 has at least a part whose width is smaller than 1 mm. Here, “width is smaller than 1 mm” means that the minimum width of the sealing part 31a formed between wrinkles of the wrinkle part 32 or between a wrinkle of the wrinkle part 32 and the proximal rim 33b or the distal rim 33a of the covering region 30 is smaller than 1 mm. In the example illustrated in FIG. 5, in the direction perpendicular to (normal direction of) the tangential direction of a unicursal line, the minimum width of the sealing part 31a formed between wrinkles of the wrinkle part 32 or between a wrinkle of the wrinkle part 32 and the proximal rim 33b or the distal rim 33a of the covering region 30 is smaller than 1 mm. In FIG. 5, a place where the width is smaller than 1 mm in the seamless sealing part 31a is indicated by an arrow.

At the place where the width of the sealing part 31a is smaller than 1 mm, the area of contact with the tracheal inner wall is also small. For this reason, the region to which pressure is applied from the cuff 3 is also small and the possibility that inflammation occurs in the tracheal inner wall is reduced. In this manner, the safety of the tracheal tube 1 is enhanced.

Note that it is preferable that the membrane thickness of the barrel portion 16 be small to facilitate formation of the place where the width is smaller than 1 mm in the seamless sealing part 31a. This is because the barrel portion 16 is deformed more readily and a region with a smaller width is readily formed in the sealing part 31a when the membrane thickness of the barrel portion 16 is smaller. Therefore, although the case in which the membrane thickness is 0.06 mm is described in the aforesaid embodiment, it is preferable that the membrane thickness be larger than 0 mm and smaller than 0.15 mm. Furthermore, it is more preferable that the membrane thickness be larger than 0 mm and equal to or smaller than 0.10 mm, and it is particularly preferable that the membrane thickness be larger than 0 mm and equal to or smaller than 0.08 mm.

To facilitate formation of the place where the width is smaller than 1 mm in the seamless sealing part 31a, it is preferable that the barrel portion 16 be composed of a soft material. This is because, when the material of the barrel portion is softer, the barrel portion 16 is deformed more readily and a region with a smaller width is readily formed in the sealing part 31a. Therefore, although the case in which the membrane of the barrel portion 16 is composed of a material having a Young's modulus of 0.34 MPa is described in the aforesaid embodiment, it is preferable that the membrane of the barrel portion 16 be composed of a material having a Young's modulus equal to or lower than 0.34 MPa.

Moreover, regarding the taper angle θ (see FIG. 3) formed by the taper part 60 and the center axis line O1, it is preferable that the taper angle θ be equal to or smaller than 40°. Furthermore, it is more preferable that the taper angle θ be equal to or smaller than 25° and it is particularly preferable to set the taper angle θ to 10° to 12°.

Although the present disclosure is described on the basis of the drawings and embodiment examples, it should be noted that it is easy for those skilled in the art to make various modifications and alterations on the basis of the present disclosure. Therefore, it should be noted that these modifications and alterations are included in the scope of the present disclosure.

For example, the distal connected part 14a and the proximal connected part 14b illustrated in FIG. 3 and FIG. 4 are fixed to the outer wall of the tube main body 2 in the state of extending to the outside of the barrel portion 16 in the center axis line direction A. However, they are not limited to this configuration. The distal connected part 14a and the proximal connected part 14b may be fixed to the outer wall of the tube main body 2 in the state of being folded into the side of the barrel portion 16 in the center axis line direction A. Furthermore, it is also possible to employ a configuration in which either one of the distal connected part 14a and the proximal connected part 14b is fixed to the outer wall of the tube main body 2 in the state of being folded into the side of the barrel portion 16 in the center axis line direction A and the other is fixed to the outer wall of the tube main body 2 in the state of being extended to the outside of the barrel portion 16 in the center axis line direction A.

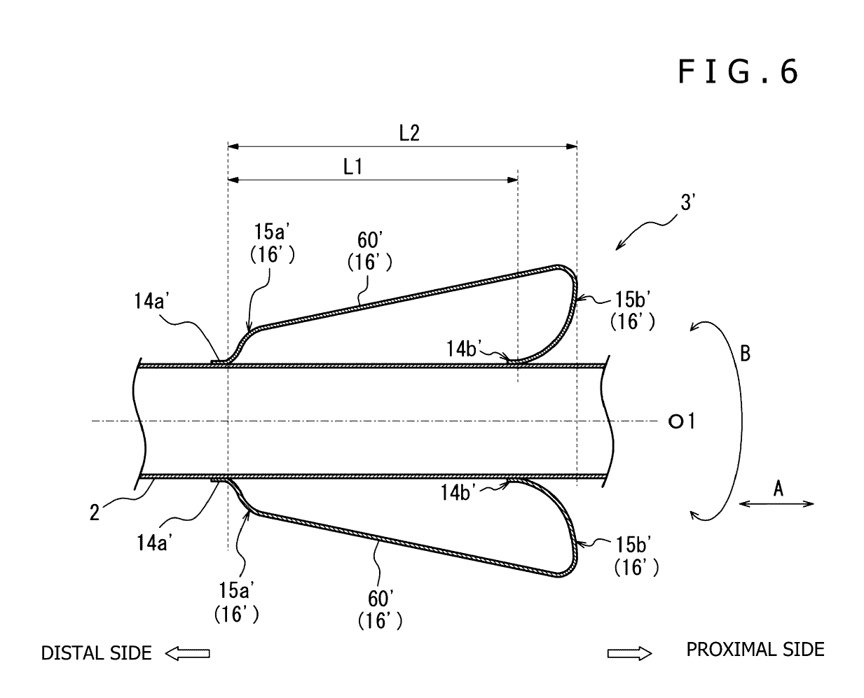

However, it is preferable that at least one of the distal connected part 14a and the proximal connected part 14b be fixed to the outer wall of the tube main body 2 in the state of being folded into the side of the barrel portion 16 in the center axis line direction A. One example of this configuration will be described with reference to FIG. 6. FIG. 6 is a sectional view illustrating a cuff 3′ as a modification example of the cuff 3. FIG. 6 illustrates the cuff 3′ in the inflated state and the section illustrated in FIG. 6 is a section including the center axis line O1 as with FIG. 4. As illustrated in FIG. 6, the distal connected part 14a′ of the cuff 3′ is fixed to the outer wall of the tube main body 2 in the state of extending to the outside of the barrel portion 16′ in the center axis line direction A, and the proximal connected part 14b′ of the cuff 3′ is fixed to the outer wall of the tube main body 2 in the state of being folded into the side of the barrel portion 16′ in the center axis line direction A.

The configuration of the distal connected part 14a′ is the same as the distal connected part 14a of the above-described cuff 3 and therefore description thereof is omitted here. The proximal connected part 14b′ is formed by fixing the end edge part continuous with the proximal side of the barrel portion 16′ in the center axis line direction A to the outer wall of the tube main body 2 in the state in which the end edge part is folded into the side of the barrel portion 16′, i.e. the distal side. If the proximal connected part 14b′ is fixed to the outer wall of the tube main body 2 in this manner, the proximal coupling part 15b′ readily projects into a projecting shape from the boundary between the proximal connected part 14b′ and the proximal coupling part 15b′ toward the proximal side in the center axis line direction A due to the elastic performance of the thin membrane member itself forming the cuff 3′. For this reason, in the section (see FIG. 6) including the center axis line O1, length L1 in the center axis line direction A between the position across which the barrel portion 16′ and the distal connected part 14a′ are continuous and the position across which the barrel portion 16′ and the proximal connected part 14b′ are continuous readily becomes shorter than maximum length L2 of the barrel portion 16′ in the center axis line direction A.

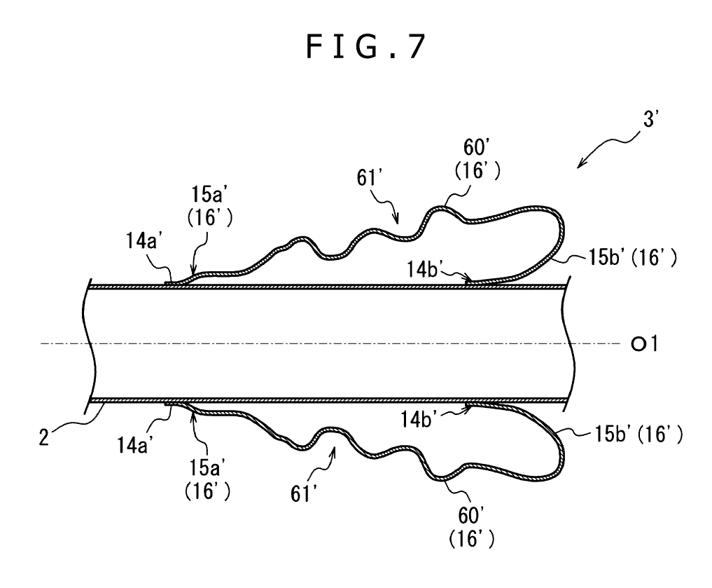

FIG. 7 is a sectional view illustrating the cuff 3′ in the middle of inflation in deformation from the deflated state to the inflated state illustrated in FIG. 6. If the configuration in which the length L2 is longer than the length L1 as described above is employed, in deforming the barrel portion 16′ from the deflated state to the inflated state, slack that waves in the center axis line direction A is readily formed in the barrel portion 16′ in the middle of inflation as illustrated in FIG. 7. In other words, when the barrel portion 16′ is inflated, groove parts 61′ that extend in the circumferential direction B are readily formed in the outer wall of the taper part 60′. By causing the outer circumferential surface of the taper part 60′ to abut against a tracheal inner wall in the state in which the groove parts 61′ are formed in the outer wall of the taper part 60′, wrinkles that are based on the groove parts 61′ and extend in the circumferential direction B are readily formed in the covering region 30 (see FIG. 5). That is, among the first wrinkles and the second wrinkles in the covering region 30, wrinkles that extend toward the circumferential direction B relatively (in FIG. 5, the second wrinkle 35 and the first wrinkle 36) are readily formed due to the above-described groove parts 61′, and wrinkles that extend toward the center axis line direction A relatively (in FIG. 5, the first wrinkle 34 and the second wrinkle 37) are readily bent, narrowed, split, or the like, which suppresses formation of the penetrating wrinkle.

Note that, in the cuff 3′ illustrated in FIG. 6 and FIG. 7, the magnitude relationship between the length L1 and the length L2 is realized by making the state in which the proximal connected part 14b′ is folded into the side of the barrel portion 16′. However, the configuration is not limited to that illustrated in FIG. 6 and FIG. 7 as long as it is a configuration in which the length L2 is longer than the length L1. However, L1<L2 can be realized with a simple configuration by making the state in which one of the distal connected part 14a′ and the proximal connected part 14b′ is folded into the side of the barrel portion 16′ as illustrated in FIG. 6 and FIG. 7. Furthermore, in the case of employing the configuration in which only one of the distal connected part and the proximal connected part is folded into the barrel portion side, it is preferable to employ a configuration in which the proximal connected part 14b′ is folded into the side of the barrel portion 16′ as illustrated in FIG. 6 and FIG. 7. This makes it easier to bring the position of the suction port 12b closer to the barrel portion 16′ in the center axis line direction A compared with the configuration in which the distal connected part is folded into the barrel portion side, and can realize a configuration in which the efficiency of suction of the foreign object X (see FIG. 1) from the suction port 12b is improved.

Here, it is particularly preferable to employ a configuration in which, in the middle of inflation of the barrel portion 16′, a length relationship of L1<L2 is satisfied in the state in which the distal coupling part 15a′ projects to the distal side in the center axis line direction A relative to the position across which the barrel portion 16′ and the distal connected part 14a′ are continuous and the proximal coupling part 15b′ projects to the proximal side in the center axis line direction A relative to the position across which the barrel portion 16′ and the proximal connected part 14b′ are continuous. If such a configuration is employed, slack is generated more readily and the groove parts 61′ are formed more readily in the taper part 60′ of the barrel portion 16′ in the middle of inflation. Note that the connection configuration of the distal connected part and the proximal connected part is not particularly limited as long as it can realize such a configuration. For example, connected parts fixed in the state of extending to the outside relative to the barrel portion 16 in the center axis line direction A like the distal connected part 14a and the proximal connected part 14b illustrated in FIG. 3 and FIG. 4 may be employed. However, if both of the distal connected part and the proximal connected part are fixed to the outer wall of the tube main body in the state of being folded into the barrel portion side, it is possible to facilitate realization of the above-described state in which the distal connected part and the proximal connected part project outward in the center axis line direction of the tube main body in the middle of inflation of the barrel portion. Furthermore, as described above, if the proximal connected part is folded into the barrel portion side and is fixed, it becomes easier to bring the position of the suction port on the proximal side relative to the cuff closer to the barrel portion in the center axis line direction of the tube main body, and it is possible to realize a configuration in which the efficiency of suction of the foreign object X (see FIG. 1) from the suction port 12b is improved.

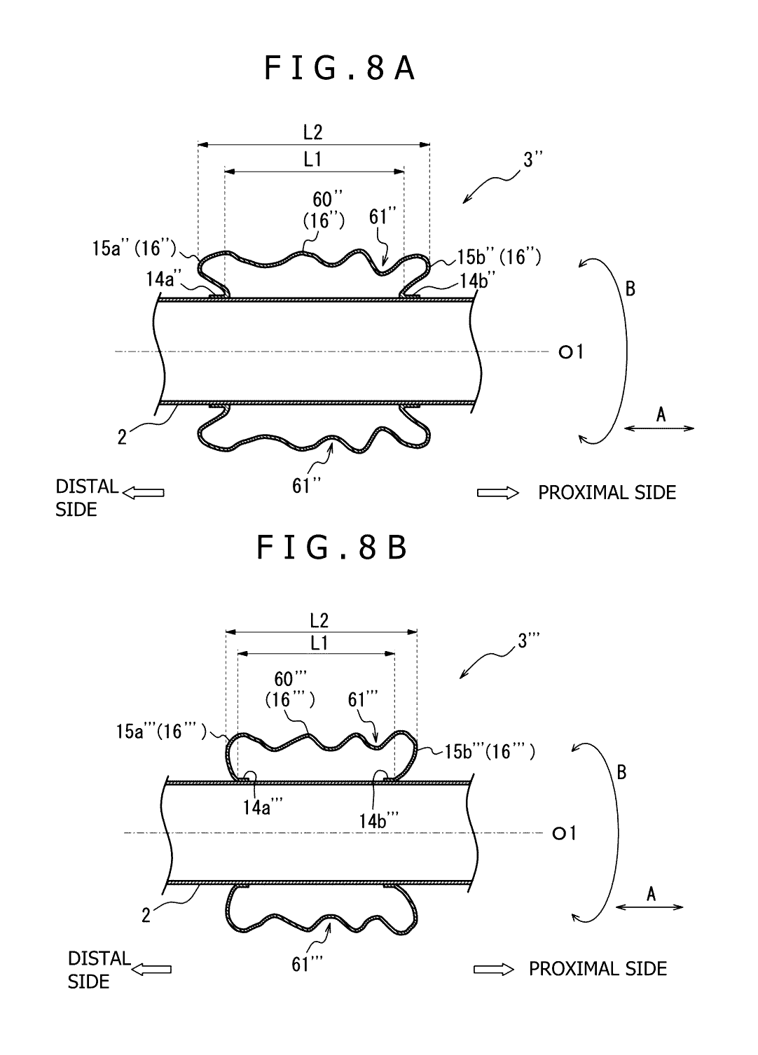

Specifically, configurations illustrated in FIG. 8A and FIG. 8B are cited as one example. FIG. 8A illustrates a cuff 3″ as another modification example of the cuff 3 of the present embodiment, and FIG. 8B illustrates a cuff 3′″ as yet another modification example of the cuff 3 of the present embodiment. FIG. 8A and FIG. 8B are sectional views illustrating the states of the cuff 3″ and the cuff 3′″ in the middle of inflation. In the cuff 3″, the state in which the distal coupling part 15a″ projects to the distal side in the center axis line direction A relative to the position across which the barrel portion 16″ and the distal connected part 14a″ are continuous and the proximal coupling part 15b″ projects to the proximal side in the center axis line direction A relative to the position across which the barrel portion 16″ and the proximal connected part 14b″ are continuous in the middle of inflation of the barrel portion 16″ can be realized by a configuration in which the distal connected part 14a″ and the proximal connected part 14b″ are fixed in the state of extending to the outside relative to the barrel portion 16″ in the center axis line direction A. Furthermore, in the cuff 3′″, the state in which the distal coupling part 15a′″ projects to the distal side in the center axis line direction A relative to the position across which the barrel portion 16′″ and the distal connected part 14a′″ are continuous and the proximal coupling part 15b′″ projects to the proximal side in the center axis line direction A relative to the position across which the barrel portion 16′″ and the proximal connected part 14b′″ are continuous in the middle of inflation of the barrel portion 16′″ can be realized by a configuration in which the distal connected part 14a′″ and the proximal connected part 14b′″ are fixed in the state of being folded into the side of the barrel portion 16′″ in the center axis line direction A. As illustrated in FIG. 8A and FIG. 8B, in the cuff 3″ and the cuff 3′″, the groove parts 61″ and 61′″ are readily formed in the outer walls of the taper parts 60″ and 60′″ in the middle of inflation.

Moreover, in the aforesaid embodiment, the case is described in which the barrel portion 16 of the cuff 3 has the taper part 60 whose outer diameter gradually increases from the side of the distal end 5 toward the side of the proximal end 6 in the center axis line direction A. However, the shape of the barrel portion 16 is not limited to the shape of the barrel portion 16 of the above-described cuff 3. The shape of the barrel portion of the cuff can be set to any shape as long as the wrinkle part in the covering region has the above-described first wrinkle and second wrinkle.

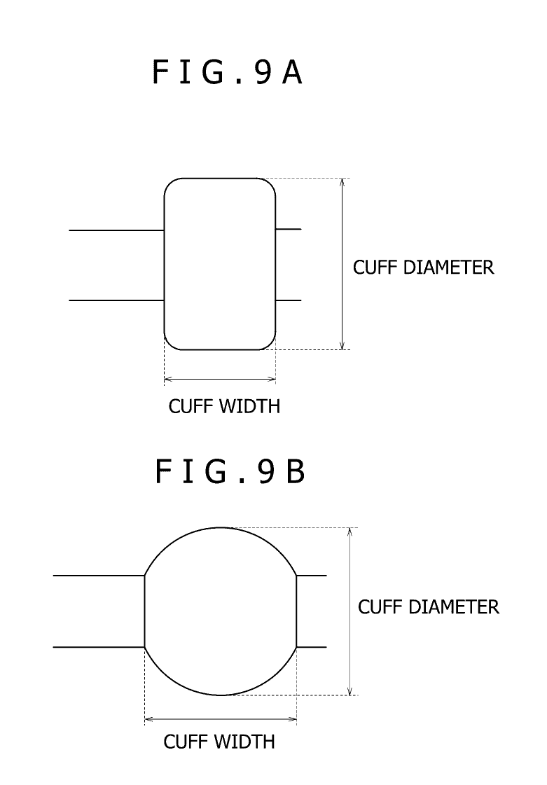

FIG. 9 is a diagram illustrating two modification examples of the shape of the cuff 3 included in the tracheal tube 1. A first modification example has a substantially circular column shape whose outer diameter is larger than the diameter of the tube main body as illustrated in FIG. 9A, and this type will be referred to as the tire type hereinafter. The cuff of the tire type has a straight line part parallel to the tube main body in side view in the barrel portion of the cuff. A second modification example has a substantially spherical shape as the outer shape as illustrated in FIG. 9B, and this type will be referred to as the spherical type hereinafter. In the cuff of the spherical type, the barrel portion of the cuff has a curved surface in side view.

Also in the cases in which the shape of the barrel portion of the cuff is the tire type and the spherical type, the similar effects as the tracheal tube 1 of the aforesaid embodiment described by taking the barrel portion 16 having the above-described taper part 60 as an example can be achieved if the wrinkle part of the covering region has the above-described first wrinkle and second wrinkle.

However, if the configuration having the taper part 60 whose diameter decreases from one side toward the other side in the center axis line direction A like the above-described cuff 3 is employed, plural wrinkles formed on the outer wall of the taper part 60 readily extend in such a manner as to get closer to each other as the position moves from one side toward the other side in the center axis line direction A, which facilitates formation of the above-described first wrinkle and second wrinkle, compared with the cuff that does not have a taper shape like the tire type.

Moreover, if only the taper part 60 whose diameter decreases from one side toward the other side in the center axis line direction A is employed as the part that becomes a taper shape at the time of inflation as in the above-described cuff 3 and a configuration that does not include a taper part whose diameter decreases from the other side toward the one side is made, it is easy to ensure the length, in the center axis line direction A, of one taper part 60 whose diameter decreases from the one side toward the other side compared with a configuration having two taper parts in which the diameter decreases from one side toward the other side and from the one side toward the other side like the spherical type. Thus, plural wrinkles that are formed on the outer wall of the taper part 60 at the time of inflation in a trachea and extend in such a manner as to get closer to each other as the position moves from one side toward the other side in the center axis line direction A can be easily brought closer to each other at a shorter distance, and the degree of interference between the first wrinkle and the second wrinkle can be enhanced.

Furthermore, the above-described cuff 3 has the rotationally-symmetric configuration about the center axis line O1 of the tube main body 2. However, it suffices that the cuff 3 be rotationally symmetric about a predetermined axis while its diameter decreases in such a manner that the outer surface of the taper part gets closer to the center axis line O1 as the position moves from one side toward the other side in the center axis line direction A. The cuff 3 is not limited to that in which the axis of the rotational symmetry corresponds with the center axis line O1.

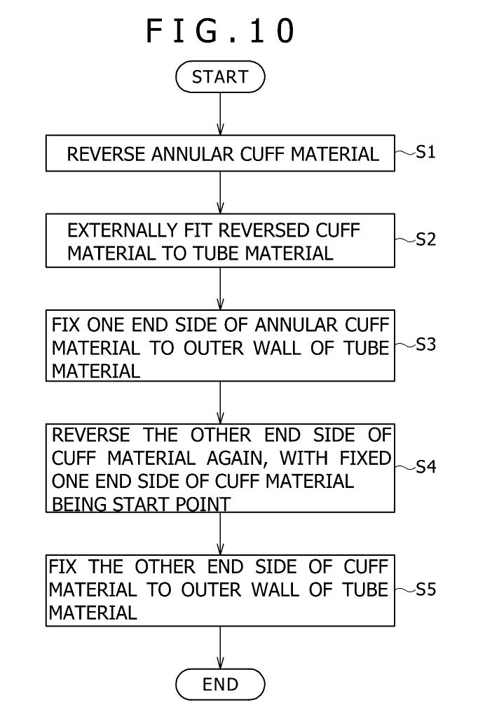

Lastly, a mounting method of the cuff 3′ of the tracheal tube 1 will be described. FIG. 10 is a flowchart illustrating the procedure of the mounting method of the above-described cuff 3′ (see FIG. 6 and FIG. 7).

As illustrated in FIG. 10, the mounting method of the cuff 3′ includes a step S1 of reversing an annular cuff material, a step S2 of inserting a tube material into the reversed cuff material to externally fit the reversed cuff material to the tube material, and a step S3 of fixing one end side of the annular cuff material to the outer wall of the tube material. The mounting method further includes a step S4 of reversing the other end side of the cuff material again, with the fixed one end side of the cuff material being the start point of the reversing, and a step S5 of fixing the other end side of the cuff material to the outer wall of the tube material. Note that the tube material means the completed tube main body 2 or what serves as the base of the tube main body 2. Furthermore, the annular cuff material means a component that forms the cuff 3′ of the tracheal tube 1 by being mounted to the tube material and serves as the base of the cuff 3′. Moreover, the annular cuff material includes a large-diameter part to become the barrel portion 16′ and end edge parts that are continuous with both sides of this large-diameter part and have a diameter smaller than the large-diameter part. The respective steps will be described in detail below.

In the step S1, the annular cuff material is reversed so that the surface to become the inner wall of the barrel portion 16′ in the large-diameter part may be exposed to the outside. However, the step S1 can be omitted if such a distinction between the front and back sides does not exist in the annular cuff material or if the cuff material has been already reversed.

In the step S2, the reversed annular cuff material is externally fitted from one end side of the tube material (for example, one end side to become the proximal end 6 of the tube main body 2). Note that the reversed annular cuff material may be externally fitted from the other end side of the tube material (for example, one end side to become the distal end 5 of the tube main body 2).

In the step S3, the end edge part to become the proximal connected part 14b′ as the end edge part on one end side of the annular cuff material externally fitted to the tube material is fixed to the outer wall of the tube material by fusion bonding or the like. Note that, when being fixed to the outer wall of the tube material, the end edge part on the one end side of the cuff material is in the state of extending to the outside of the large-diameter part in the center axis line direction of the tube material (same direction as the center axis line direction A of the tube main body 2). Furthermore, although being the end edge part to become the proximal connected part 14b′, the end edge part on the one end side fixed to the tube material is located on the distal side of the tube material relative to the large-diameter part, i.e. on the side to become the side of the distal end 5 of the tube main body 2, because the cuff material is in the reversed state when this end edge part on the one end side is fixed to the outer wall of the tube material.

In the step S4, the other end side of the cuff material is reversed again, with the fixed end edge part on the one end side of the cuff material being the start point of the reversing. That is, the other end side is reversed so that the surface to become the outer wall of the barrel portion 16′ in the large-diameter part may be exposed to the outside. More specifically, with the proximal connected part 14b′ being the start point of the reversing, the end edge part to become the distal connected part 14a′ as the end edge part on the other end side of the annular cuff material and the large-diameter part to become the barrel portion 16′ are reversed. This causes the proximal connected part 14b′ to become the state of being folded into the side of the large-diameter part to become the barrel portion 16′ in the center axis line direction of the tube material. Meanwhile, the end edge part to become the distal connected part 14a′ becomes the state of extending to the outside of the large-diameter part to become the barrel portion 16′, specifically to the distal side of the tube material relative to the large-diameter part, in the center axis line direction of the tube material.

In the step S5, the end edge part to become the distal connected part 14a′ as the end edge part on the other end side of the cuff material is fixed to the outer wall of the tube material by fusion bonding or the like. This completes the fixing of the cuff material to the tube material.

In the above-described manner, the cuff 3′ can be mounted to the tube material. Note that, although the mounting method of the cuff 3′ is described with reference to FIG. 10 here, in the case of the cuff 3 illustrated in FIG. 3 and FIG. 4, it suffices to carry out a step of externally fitting an annular cuff material to become the cuff 3 to a tube material without reversing the cuff material and fixing one end side of the annular cuff material to the outer wall of the tube material and a step of fixing the other end side of the cuff material to the outer wall of the tube material. More specifically, both end edge parts that extend to the outside of the large-diameter part to become the barrel portion 16 in the center axis line direction of the tube material in the annular cuff material externally fitted to the tube material are fixed to the outer wall of the tube material by fusion bonding or the like, to form the distal connected part 14a and the proximal connected part 14b.

The present disclosure relates to a tracheal tube.

Having described the preferred embodiment of the present disclosure with reference to the accompanying drawings, it is to be understood that the disclosure is not limited to the precise embodiment and that various changes and modifications could be effected therein by one skilled in the art without departing from the spirit or scope of the disclosure as defined by the appended claims.

What is claimed is:

1. A tracheal tube comprising:
a tube main body; and
a cuff that is mounted to the tube main body and has a barrel portion capable of being deflated and inflated, wherein
a covering region that covers a tracheal inner wall of a trachea in a state in which the barrel portion is inflated in the trachea has a contact part that gets contact with the tracheal inner wall and a wrinkle part that does not get contact with the tracheal inner wall, and the wrinkle part includes a first wrinkle and a second wrinkle that extends in a direction intersecting a predetermined part of the first wrinkle and has a termination part that does not intersect the predetermined part and terminates at such a position as to sandwich the contact part with the predetermined part.

2. The tracheal tube according to claim 1, wherein the termination part extends toward a circumferential direction of the tube main body relative to an extension direction at the predetermined part of the first wrinkle.

3. The tracheal tube according to claim 2, wherein at least one end of the first wrinkle reaches a distal rim or a proximal rim of the covering region.

4. The tracheal tube according to claim 1, wherein the termination part extends toward a center axis line direction of the tube main body relative to the extension direction at the predetermined part of the first wrinkle.

5. The tracheal tube according to claim 4, wherein one end of the second wrinkle reaches a distal rim or a proximal rim of the covering region.

6. The tracheal tube according to claim 1, wherein minimum width of the contact part between the first wrinkle and the termination part of the second wrinkle is smaller than 1 mm.

7. The tracheal tube according to claim 1, wherein the contact part has a sealing part that gets contact with the tracheal inner wall seamlessly in a circumferential direction of the barrel portion, and width of at least part of the sealing part is smaller than 1 mm.

8. The tracheal tube according to claim 1, wherein the barrel portion is rotationally symmetric about a predetermined axis in the inflated state and has a taper part whose diameter around the predetermined axis increases from one end side of the predetermined axis toward the other end side.
